(19) United States
(12) Patent Application Publication
(10) Pub. No.: US 2018/0179573 A1
(43) Pub. Date: Jun. 28, 2018

(54) Examination of a Sterilization Effect

(71) Applicants: Niwin Flier, Düren (DE); Klaus Haberer, Hofheim (DE); Alexander Böker, Berlin (DE)

(72) Inventors: Niwin Flier, Düren (DE); Klaus Haberer, Hofheim (DE); Alexander Böker, Berlin (DE)

(73) Assignee: Compliance Advice and Services in Microbiology GmbH, Köln (DE)

(21) Appl. No.: 15/841,664

(22) Filed: Dec. 14, 2017

(30) Foreign Application Priority Data
Dec. 14, 2016 (DE) 10 2016 124 284.5

(51) Int. Cl. C12Q 1/22

(52) U.S. Cl. CPC C12Q 1/22

(57) ABSTRACT

Provided is an apparatus to verify a sterilisation effect. The apparatus includes a carrier element, a bonding agent, and a population of microorganisms. At least two sub-areas of the carrier element are bondable and are spaced apart. The bondable sub-areas of the surface due to the arrangement of the bonding agent are functionalisable. The bonding agent binds to the bondable sub-area of the surface and provides free adhesive spots for microorganisms to be adhered so that a stable intermediate bond is creatable between the bondable sub-area and microorganisms to be adhered. The microorganisms of the population are purified and excess biological material attaching to the microorganisms is removed. The respective microorganisms have an almost single-cell character. A purified population of microorganisms is adherable to one of the functionalised sub-areas of the surface so that the microorganisms are adhered in a high areal density and within a single layer.

### CROSS-REFERENCE TO RELATED PATENT APPLICATION

This patent application claims priority to German Application No. 10 2016 124 284.5, filed Dec. 14, 2016, the entire teachings and disclosure of which are incorporated herein by reference thereto.

### FIELD

The invention relates to an apparatus for examination of a sterilisation effect. Furthermore, the invention relates to a method for its manufacture and a use of the apparatus according to the invention.

### BACKGROUND

Products that must be sterile to ensure their application reliability (e.g. for sterile pharmaceutical products and medical devices), or to ensure their shelf-life (e.g. in the case of preserves) must undergo an appropriate sterilisation process. Depending on the properties of the sterilised material, different principles are used, such as, for example, exposure in saturated steam, dry heat, or ionising radiation. Furthermore, chemical sterilisation is used, above all, for the sterilisation of surfaces, for example, by means of hydrogen peroxide (H₂O₂) steam in insulators or by means of gassing with ethylene oxide (C₂H₄O) in the case of medical equipment and disposable products.

For examination of the biological effectiveness of the sterilisation method, continuous forms, for example, spores of certain microorganisms with a high resistance to the sterilisation method are generally used as a bioindicator. The resistance of the microorganisms can always only be determined with relation to a sterilisation method and is specified as decimal reduction time (D-value). This D-value is the constant time, in which a population treated in this manner is reduced by 90% under the given sterilisation conditions. On the one hand, the D-value is a specific property of the strain of the microorganisms, for example, a strain of bacteria, wherein cultivation, sporulation, and the preparation of spores of the microorganisms not only modulate the D-value of each batch, but also, the resistance of the individual microorganisms is subject to a fluctuation range, which is dependent on the manufacturing process. Therefore, D-values are inevitably tainted with a biological uncertainty. On the other hand, the reduction time changes with different effectiveness of the sterilisation process. Therefore, the indicated D-value always only applies to the related sterilisation conditions.

In order to check the sterilisation effect, the populations of microorganisms are applied to appropriate carriers in high quantities. The carriers prepared in this manner are then subject to the sterilisation conditions to be verified in a sterilisation chamber with or without sterilisation material. The D-value can be calculated from the knowledge of the number of microorganisms on the carrier prior to the exposure and the number of surviving microorganisms after the exposure. In practice, the determination of the explicit number of surviving microorganisms is possible, but associated with high overhead, and also error-prone due to the required recovery of the microorganisms. Therefore, the average time until the complete inactivation of the spores of the original populations on each of the established carriers is estimated.

For evaluation, the carriers are put into contact with a nutrient medium after exposure to sterilisation conditions and then are subjected to previously defined growth conditions. Thereby, the last surviving spores of the population of microorganisms should be made detectable. An extended time to complete eradication shows a reduced effectiveness of the sterilisation process, and vice versa. A significant issue in this evaluation is the type of mortality curve, which asymptotically approaches the zero value as first-order kinetics. The time of complete inactivation cannot be determined exactly, but requires evaluation with the help of a statistical method. The actual original number of microorganisms on a carrier, as well as the inevitable biological vagueness of the D-value make a statistical approach mandatorily necessary.

In the case of the currently known bioindicators, it is specified that a single population of about 1×10⁶ microorganisms is arranged on each carrier. This population of microorganisms occupy all or most of the entire surface of the carrier (examples are paper strips approximately 3×0.5 mm or 3×0.5 cm for steam sterilisation or round steel carriers with a diameter of about 0.8 cm for gas sterilisation). The ISO standard requires that the manufacturer's specifications for the number of microorganisms per carrier should be reproducible by between −50% and +300%. Furthermore, the D-value indicated by using the method of the manufacturer of this bioindicator should be reproducible by ±20%. It is obvious that the uncertainty associated with these margins is large.

Therefore, the survival of individual spores after a recent exposure to sterilisation conditions may be able to be explained from the variability of biological indicators, as well as from a low effect of the sterilisation procedure.

According to the Ph. Eur. (Pharmacopoea Europaea, European Pharmacopoeia) bioindicators are proposed, where, after exposure to the sterilisation conditions of a standard sterilisation procedure, the likelihood of finding surviving cells is 1 in 10⁴. If surviving microorganisms exist after exposure, this is considered evidence of inadequate sterilisation, however, if all microorganisms are completely eradicated, the assertion remains concerning the correct effectiveness of the process, even though it is weak, since it is only comprehensible that all microorganisms were eradicated, not however, for example, how long it lasted or how resistant the microorganisms were to the sterilisation conditions.

### SUMMARY OF SEVERAL EXEMPLARY EMBODIMENTS

Based on this prior art, it is the object of the invention to provide an apparatus for the examination of a sterilisation effect that improves the aforementioned problems and that makes it possible to carry out a statistically relevant evaluation.

With the invention, an apparatus for examination of a sterilisation effect is proposed as the technical solution to this object, thereby comprising:

a carrier element, wherein at least two sub-areas of the surface of the carrier element are bondable and wherein the bondable sub-areas are spaced away from each other;

a bonding agent, wherein the bondable sub-areas of the surface due to the arrangement of the bonding agent to these are functionalisable, wherein the bonding agent, on the one hand, binds to the bondable sub-area of the surface, and on the other hand, due to the respective size of the bondable sub-area, provides free adhesive spots for microorganisms to be adhered so that a stable intermediate bond is creatable between the bondable sub-area of the surface of the carrier element and microorganisms to be adhered, and

a population of microorganisms, wherein the microorganisms of the population are purified, wherein excess biological material attaching to the microorganisms is removed and the respective microorganisms have an almost single-cell character,

wherein a purified population of microorganisms is adherable to one of the functionalised sub-areas of the surface in such a way that the microorganisms are adhered in a high areal density and primarily without mutual covering within a single layer.

On the one hand, the invention is based on the knowledge that an adhesion of microorganisms at a functionalised sub-area of the surface of the carrier element makes a defined and reproducible number of microorganisms for each functionalised sub-area possible. The adhesion of microorganisms primarily without covering themselves makes a uniform exposure of individual microorganisms of a population of microorganisms to sterilisation conditions possible. Uniform access of a sterilising agent as well as, for example, for condensing water vapour, as well as for vaporous hydrogen peroxide, is possible. This makes a uniform exposure to all microorganisms that are adherable to an embodiment of an apparatus according to the invention possible. The adherable microorganism-population is optimised for the sterilisation effect to be characterized in numbers and resistance in such a way, that, in the case of the sterilisation effect to be expected, all or almost all populations of the populations that are adherable to the carrier element can be fully eradicated. A reduced sterilisation effect is indicated by the survival of the microorganisms of an individual population of microorganisms of the embodiment of an apparatus according to the invention. Due to the high areal density in a population of microorganisms according to the invention combined with the resistance of the preparation prior to the adhesion of microorganisms, the sterilisation effect load can be chosen accordingly in order to make a statistically significant assertion.

On the other hand, the invention is based on the knowledge that the statistic evaluation of a plurality of microorganism populations is considerably simplified in case a plurality of populations of microorganisms can be exposed to sterilisation conditions at the same time using a single apparatus or a single carrier element only. According to the invention, it is also provided that at least two populations of microorganisms are situated or adhered on only a single carrier element. This allows for statistical evaluation of the sterilisation when only using one apparatus according to the invention.

In one embodiment according to all the aforementioned aspects of the invention, the microorganisms of one population are adherable to a bondable and functionalised sub-area on the surface of the carrier in a defined manner, wherein a separation of individual populations on only a single carrier element is possible. By means of this, the predominant sterilisation conditions across populations is primarily uniform, whereby a much improved statistical evaluation is made possible, wherein false positive and false negative outliers are minimised in the evaluation result.

Within the meaning of the present invention, under the term bonding agent, a substance is understood that makes a bonding between two immiscible substances possible. The bonding agent is arranged on a surface of a first substance and enables a close, stable and permanent bond between the first substance and another second substance.

Within the meaning of the invention, a population of microorganisms comprises a number of microorganisms that have resistance to a sterilisation principle and the sterilisation conditions associated therewith. Microorganism-populations in quantities of approximately 10⁴, approximately 10⁵, approximately 10⁶, approximately 10⁷ or approximately 10⁸ microorganisms per population are adherable to a bondable and functionalised sub-area of the surface of the apparatus according to the invention.

Preferably, the carrier element has two to 96, four, six, eight, ten, twelve, 20, 21, 22, 24, 27, 30, 40, 48 or 96 activated and/or functionalised sub-areas for the adhesion of microorganisms. The carrier element may have more than two activated sub-areas. According to the invention, the number of populations on activated sub-surfaces is at least two, a higher number of populations allows for improved statistical evaluation. The upper limit of the number of populations is given by the described space requirement of the individual population plus a sufficient physical distance between all of the populations, which makes an independent evaluation of all individual populations possible. The number of bondable sub-areas of the surface of the carrier element is limited by means of i) the size of the carrier element, ii) the required size of an individual population for a significant interpretability, iii) by means of the obtainable areal density with the adhesion of microorganisms that has a direct effect on the size of the required bondable sub-area, as well as iv) the arrangement of populations on the carrier element considering the separation of the individual populations of microorganisms. Multiple populations on a single carrier element of the apparatus only enable a joint exposure to sterilisation conditions occurring during a sterilisation procedure. The evaluation result within the scope of the statistical evaluation is considerably less sensitive to outliers due to the joint exposure of the multiple populations, since the predominant sterilisation conditions are considerably more constant.

The spacing of the at least two bondable sub-areas is provided by establishing a physical distance between these bondable sub-areas of the surface of the carrier element, wherein no contact points may exist between the two bondable sub-areas of the surface of the carrier element. Preferably, the physical distance is so big that the viability of individual microorganisms in the microorganism-populations adhered to the bondable sub-areas can be made detectable independently and separate of each other. Otherwise, individual microorganism-populations could mutually contaminate each other so that individual surviving microorganisms of the respective population can no longer reliably be assigned to a specific population after carrying out a sterilisation. For example after carrying out a sterilisation, a population of microorganisms is fully eradicated and in contrast to this, another population is not. In the case of a connection between both microorganism-populations, individual microorganisms could “migrate” in such a way that reliable assignment of the individual surviving microorganisms into their respective population is not possible. This leads to a false positive or false negative result of statistical evaluation.

Preferably, the surface of the bondable sub-areas of the surface is fully and evenly covered with a bonding agent respectively so that microorganisms are adherable onto the entire surface of the respective bondable sub-area of the surface of the carrier element. Thereby, a bondable sub-area bonds with the bonding agent with such a high areal density that microorganisms are adherable to the carrier element at the entire bondable sub-area in the density predetermined by the free adhesive spots. The number of free adhesive spots, also called bonding spots, can, on the one hand, be modulated by the surface of a bondable sub-area, and, on the other hand, by the areal density of the bonding agent.

In an embodiment of the present invention according to all the aforementioned aspects, adhesion of microorganisms primarily occurs, in particular, only at the bondable and functionalised sub-areas of the surface of the carrier element. By means of this, the adhesion of at least two populations of microorganisms on only a single carrier element according to an embodiment of an apparatus according to the invention according to all of the aforementioned aspects is possible.

According to the invention, microorganisms to be adhered are purified before their adhesion to a carrier element according to one of the aforementioned embodiments of the apparatus. During this purification, also called cleaning, the microorganisms are separated from biological material, such as nutrient media components or cell debris, which has been caused by the cultivation of the microorganisms. For example, the purification comprises wash cycles and centrifugation steps, which, for example, enable a chemical and/or physical purification. A possible purification of microorganisms is indicated in the following. According to the invention, a high level of purification of the microorganisms to be adhered is provided, wherein such a highly purified preparation of microorganisms has been purified so intensively that also loose foreign elements connecting to the surface of the microorganisms are removed. In terms of the invention, this feature of such a purified microorganism is comprehended under an microorganism that has a single-cell character. The resistance properties of the microorganisms to sterilisation are thereby maintained, wherein this can be ensured by means of a related test.

This enables that only the adhesion of highly purified microorganisms onto the free adhesive spots of the bonding agent primarily takes place. A clumping and/or secondary attachment of individual microorganisms, in particular, caused by a cross-connection via contaminating biological material, is avoided. Furthermore, this supports the adhesion to the bondable and functionalised sub-areas of the surface of the carrier element in a high areal density.

A preferred embodiment of the invention is characterized in that the high areal density of the microorganisms, which are adherable to the functionalised sub-areas of the surface of the carrier element, corresponds to a value, the standard deviation of which is approximately 10% or less with reference to a areal density of approximately 10⁶ microorganisms per 5 mm². Preferably, the standard deviation is approximately 9% or less, approximately 8% or less, approximately 7% or less, approximately 6% or less, approximately 5% or less, approximately 4% or less, preferably approximately 3% or less, especially preferred approximately 2% or less or approximately 1% or less with reference to a areal density of approximately 10⁶ microorganisms per 5 mm². If a population of microorganisms is adhered to a bondable and functionalised sub-area of the surface of the carrier element, the absolute individual quantity of microorganisms of which deviates by approximately 10⁶ microorganisms, their required surface changes according to the indicated ration of approximately 10⁶ microorganisms per 5 mm², however, the areal density continues to correspond to a value, the standard deviation of which is approximately 10% or less with reference to a areal density of approximately 10⁶ microorganisms per 5 mm², wherein other standard deviations, which have been previously indicated, are also possible. The maximum obtainable areal density in the case of an adhesion of microorganisms to an embodiment according to all the aforementioned aspects of the present invention is 10⁶ microorganisms per mm² in the case of a constant single-cell paracrystalline arrangement.

In an embodiment of the invention, the adhesion of microorganisms allows the microorganism load that should be inactivated during the process according to the requirements at hand to vary according to the process. Higher resistance or a greater number of used microorganisms allows for a meaningful verification of highly effective processes, a greater number of populations of microorganisms per carrier element enables more precise statistical evaluation. In another embodiment of the invention, the number of microorganisms of all individual populations are the same on a carrier element, wherein the standard deviation is ±10% or less, preferably the standard deviation is ±9%, ±8%, ±7%, ±6%, ±5% or less.

The requirements for the inactivation of microorganisms, which are adherable to an apparatus according to the invention result from the combination of a number of microorganisms for each population, the resistance of the respective microorganisms and the number of the used populations of microorganisms, meaning the respective populations, which are adherable to a functionalised sub-area of an apparatus according to the invention. At a sterilisation process, which expected sterilisation effect eradicates, thereby meaning should inactivate, the used microorganisms at 8 logarithmic orders of magnitude, the carrier element has 10 populations each having 10⁶ spores. The total number of microorganisms on the medium is then 10⁷=10×10⁶. The probability of survival is 10⁻¹ in the statistical expectation, thus theoretically, only one in 10 carrier elements will still have a surviving spore.

If the sterilisation effect is reduced so that only seven orders of magnitude are eradicated, the probability of survival is 10⁰=1. That means that in approximately 60% of all carrier elements, populations with surviving microorganisms are found. If only six orders of magnitude are eradicated by the process, the probability of survival in each population is 10⁰, thereby, in approximately 60% of all populations, surviving microorganisms are expected. In 100 populations each having 10⁵ spores for each carrier element, the meaningfulness of the indicator concerning the effectiveness of the process is just as high, the statistical assertion however is more precise.

As an example, the following comparison with the prior art is indicated:

It is known from the prior art, to arrange 10⁶ microorganisms on a paper strip with the size of 3 cm×0.5 cm=1.5 cm²=150 mm². This corresponds to about 10⁶ microorganisms per 150 mm², i.e. 6667 microorganisms per mm².

In addition, it is known to arrange approximately 10⁶ microorganisms on a round steel carrier with a diameter of about 0.8 cm. This corresponds to a surface of about (0.8 cm÷2)²×π=0.5 cm²=50 mm²), thus approximately 10⁶ microorganisms per 50 mm²=20000 microorganisms per mm².

The apparatus according to the invention enables to arrange 10⁶ microorganisms per 5 mm², meaning to arrange approximately 200000 microorganisms per mm².

A preferred embodiment of the invention provides that the adhesion of microorganisms in the individual layer takes place in such a way, wherein approximately 10% or less of the microorganisms are covering each other. Preferably, the standard deviation of the number of microorganisms covering each other is approximately 10% or less with reference to a surface unit of the bondable sub-area of the surface in comparison to another surface unit of the bondable sub-area of the surface. Preferably, approximately 9% or less, approximately 8% or less, approximately 7% or less, approximately 6% or less, approximately 5% or less, approximately 4% or less, preferably approximately 3% or less, especially preferred approximately 2% or less or approximately 1% or less of the microorganisms are covering each other of a by a population of microorganisms.

A preferred embodiment of the invention provides that the adhesion of microorganisms primarily takes place without covering each other in an almost monolayer, wherein no more than approximately 10% of the microorganisms cover each other. Preferably, few or none of the evaluated microorganisms are covered. For example, this may be sustained by an electron microscopic evaluation of no less than 200 microorganisms in the peripheral area of representative activated surfaces.

The arrangement in an almost monolayer, also called an individual layer, in primarily one layer is made possible by an adhesion of the microorganisms to the carrier element via the bonding agent, wherein the microorganisms are adhered almost only or only via the intermediate bonding of the bonding agent to the bondable sub-area of the carrier element. Appropriate and sufficient washing procedures of the surfaces with microorganisms applied to them remove secondarily attached microorganisms, while the microorganisms that are permanently adhered to the activated sub-area by means of the bonding agent are not washed or removed. After the washing procedure, the microorganisms that still primarily remain adhered to the bondable sub-areas via the bonding agent, which are not removable by the washing procedure, in particular only those microorganisms still remain. This considerably reduces the covering or the clumping of microorganisms. In the resulting monolayer, a reciprocal protection of the microorganisms against the sterilisation conditions is avoided to the furthest extent possible and the possibility of the reciprocal protection of the microorganisms is minimised.

A further embodiment of the invention is characterized in that the at least two bondable sub-areas of the surface of the carrier element are activated.

Under the term, activation of a surface, within the meaning of the present invention, the mechanical thermal and/or chemical treatment of the surface or a treatment of the surface along those lines is understood.

The activation of at least one sub-area of the surface of the carrier element is preferably obtained by means of plasma treatment. This increases the surface tension on the bondable sub-areas. To only activate one sub-area of the surface of the carrier element, the part which is not to be activated belonging to the surface is shielded against the entering of plasma.

An embodiment of the invention provides that the respective surface of the bondable sub-areas on the surface of the carrier element is primarily the same in size. Along with this, the bondable sub-areas, in addition to an identical surface area, may have an identical shape. Due to the identical size the bondable sub-areas, these primarily provide the same number of free adhesive spots for microorganisms to be adhered after the functionalisation using a bonding agent. In this way, it can be ensured that the same individual number of microorganisms can be adhered onto each bondable sub-area on the surface of the carrier element, which has been functionalised using the bonding agent.

In another embodiment of the invention, the bonding agent binds to the bondable sub-areas of the surface of the carrier element in electrostatic or covalent manner. Another embodiment of the invention provides that the microorganisms to be adhered bind to the adhesive spots provided by the bonding agent in an electrostatic or covalent manner.

A functionalisation due to a coating or a wetting of the bondable sub-area of the surface particularly takes place by means of applying an ionic layer. This enables the electrostatic adhesion of microorganisms, wherein an intermediate ionic bond can be established between the bondable sub-area of the surface or of the ionic layer applied to the bondable sub-area of the surface and the microorganisms to be adhered. The ionic layer is preferably implemented by means of an ionic bonding agent, such as a polyelectrolyte. Applying the ionic layer to the bondable sub-area takes place by means of wetting it with the ionic bonding agent.

Applying a covalent bonding layer particularly takes place by applying a cross-linking agent as a bonding agent, wherein the cross-linking agent is applied to a bondable sub-area of the surface. To apply the covalently binding layer or the cross-linking agent, e.g. the respective bondable sub-area of the surface can be activated beforehand in such a way that the cross-linking agent can establish a covalent bond with this sub-area of the surface of the carrier element. The adhesion of microorganisms takes place subsequently in such a way that these establish a covalent bond with the covalently binding layer or the cross-linking agent. In particular, epoxides, such as epoxysilane are suitable as cross-linking agent.

In another embodiment of the invention, the bondable sub-areas of the surface of the carrier element are primarily free of cavities. In such cavities, microorganisms may protect themselves against sterilisation conditions. A cause for the higher resistance of a population of microorganisms on a carrier element of a known bioindicator is that the surface of the carrier, on which the microorganisms are arranged, has uneven areas like e.g. cavities. Individual microorganisms lie in such a cavity in a protected manner so that this microorganism is at least partially protected against sterilisation conditions predominant during the sterilisation process due to the outer conditions of the cavity. Accordingly, according to the invention, it is provided to design the sub-area of the surface of the carrier element, on which microorganisms or a population of microorganisms are bonded, in such a way that no cavities are primarily present, in which microorganisms can protect themselves from sterilisation conditions. This is enabled, for example, by using a carrier element provided with this feature, wherein an embodiment of the invention according to the invention is indicated in the following that makes this possible.

Another embodiment according to one of the aforementioned aspects of the invention provides that the surface of the carrier element is primarily smooth. Smooth within the meaning of the invention means that no bubbles or hollows as projections or dents are present on the surface. For example, materials cast from a melt, which are made by means of a bubble-free procedure, can be used. For example, for avoiding bubbles a degassing can take place where necessary before casting. Furthermore, a carrier element can be smoothed using a surface method, for example, by highly polishing it.

Due to this smooth embodiment of the surface of the carrier element, the surface is also particularly free of cavities, in which individual microorganisms can be protected from sterilisation conditions. This makes the removal of attached microorganisms on a non-bondable sub-area of the surface of the carrier element easier, for example, by means of a washing process. Those microorganisms, that are attached to a functionalised sub-area of the surface of the carrier element cannot be removed by means of the washing process due to the permanent and stable bonding of the bonding agent on the carrier element. Thereby, it can be ensured that microorganisms are only bonded to the functionalised sub-areas by means of the specific bonding positions or adhesive spots of the bonding agent.

An embodiment of the invention provides that the carrier element is primarily plate-shaped, spherical, polyhedral or the like. Thereby, the carrier element is made of glass, plastic or the like, for example. As a material for the element, for example, silicate glass or polycarbonate is suitable to particularly implement the element with the above-mentioned features of the invention.

In another embodiment, the bondable sub-areas of the surface of the carrier element are elevated with regard to the non-bondable sub-area of the surface of the carrier element. Preferably, the bondable areas of the surface of the carrier element are arranged on the columnar ridges. The ridge of the bondable sub-areas of the surface of the carrier element reduces the risk of a migration, meaning a mutual contamination of microorganisms by the populations of microorganisms respectively attaching to the apparatus according to the invention.

In a preferred embodiment of the invention, the microorganisms are spores, in particular, endospores of the family Bacillaceae and Clostridiaceae. Preferably, the endospores of the Bacillus and Clostridium types. A plurality of these microorganisms can respectively adhere to each one of the bondable and functionalised sub-areas of the surface of the carrier element according to the invention in a unit forming a population or a colony.

An embodiment of the apparatus according to the invention provides a cover for transport protection, wherein the cover protects the microorganisms attached to the apparatus against contamination. The surrounding cover can be detachable so that this can be removed prior to carrying out sterilisation or the cover is microporous so that sterilisation agents can penetrate the cover, meaning the cover is permeable to sterilisation agents such as steam and hydrogen peroxide (H₂O₂), however not permeable to microorganisms so that a contamination of the microorganisms connected to the carrier element of the apparatus or the populations of microorganisms can be ruled out. Therefore, the surrounding cover can also provide the function of transport protection.

The apparatus according to the invention is suitable both for use in the case of thermal as well as chemical sterilisation.

Furthermore, the object according to the invention is solved by means of a method to manufacture an apparatus according to the invention, which comprises the following method steps:
Providing a carrier element, wherein at least two sub-areas of the surface of the carrier element are bondable and wherein the bondable sub-areas care spaced away from each other.
Functionalisation of the bondable sub-areas of the surface of the carrier element with a bonding agent, wherein the bonding agent, on the one hand, binds to the bondable sub-area of the surface, and on the other hand, due to the size of the bondable sub-area, provides free adhesive spots for microorganisms to be adhered so that a stable intermediate bond is created between the bondable sub-area of the surface of the carrier element and the microorganisms to be adhered;
Purification of microorganisms, wherein excess biological material attaching to the microorganisms is removed so that the microorganisms have an almost single-cell character; and
Adhesion of the microorganisms, wherein the purified microorganisms are applied to the sub-areas functionalised with the bonding agent and the microorganisms adhere to the adhesive spots provided by the bonding agent so that the microorganisms are adhered in a single layer without primarily covering each other and in a high areal density.

[symbol] are spaced away from each other. The individual method steps of the method according to the invention can, in particular, be carried out in the described way pertaining to the apparatus according to the invention.

In an embodiment of the invention, the functionalisation of at least one bondable sub-area takes place by printing with a polyelectrolyte as an ionic bonding agent. For this purpose, in particular, a type of stamp, for example a silicon stamp can be added into a polyelectrolyte solution for the duration of a few minutes and then be put into contact with at least one activated sub-area of the carrier element. Excess polyelectrolyte can preferably be washed off using a suitable means, for example, water.

The adhesion of microorganisms on at least one functionalised sub-area can, for example, take place, in particular, by means of an electrostatic adhesion, for example, to the numerous primary and secondary amino groups of the polyelectrolytes. For example, these support dissociable groups. These groups are protonated when adding water, forming a strong alkaline polycation. The polycation may, for example, electrostatically bind to the anions of the surface of a microorganism. Preferably, the electronic potential at the surface of the microorganisms to be adhered is high with a zeta potential of the microorganisms of e.g. −60 mV, wherein anions are achieved on their surface particularly by suspending them in deionized water, reaching a pH-level of 6.

An embodiment according to one of the aforementioned aspects is characterized by the following method step:
Purification of the carrier element, wherein secondarily attached microorganisms and/or microorganisms attached on a non-bondable sub-area of the carrier element are removed.

Here, a solvent, such as 2-propanol, is suitable. In addition, a purification of the carrier element can alternatively or additionally take place in an ultrasonic bath.

A further embodiment of the invention according to the aforementioned aspects is characterized in that the at least two bondable sub-areas are activated prior to functionalisation, wherein the activation preferably takes place by means of a plasma treatment.

Preferably, the carrier element can be purified before activation.

The plasma treatment increases the surface tension on the bondable sub-area of the carrier element. The plasma treatment can preferably take place in a plasma furnace, such as, for example, a vacuum chamber with glow discharge, wherein a sub-area of the surface of the carrier element that is not to be activated is covered, for example by means of a silicone template or shadow mask. This effectively prevents activation of this covered sub-area of the surface of the carrier element. For activation, the carrier element is, for example treated with oxygen plasma, preferably, at about 0.1 mbar for a period of about 1 min. until about 10 min. The plasma treatment causes some carbon groups of the carrier element to oxidize, wherein oxygen atoms accumulate and form reactive active hydroxyl groups.

A further embodiment of the invention is characterized in that the adhesion of microorganisms occurs by microorganisms to be adhered being applied in excess to at least one functionalised sub-area of the surface of the carrier element.

A further embodiment of the invention provides that the adhesion of microorganisms occurs by microorganisms to be adhered, for example as a suspension, being applied in excess to at least one functionalised sub-area of the surface of the carrier element. The bonding between a microorganism and an adhesion provided by the bonding agent to the functionalised sub-area is stronger than the bonding of the microorganisms among each other. This allows an adhesion of microorganisms only in the adhesive spots of the bonding agent to this functionalised sub-area of the carrier element. Due to the chemical purification of the microorganisms and the presence of microorganisms to be adhered with a single-cell character, a high level of areal density of the microorganisms in almost a monolayer at the functionalised sub-area of the surface of the carrier element can be achieved.

Furthermore, the object of the invention is solved by using the apparatus according to the invention to examine a sterilisation effect and/or as a bioindicator.

The following explains additional details, features and advantages of this invention with the aid of an exemplary embodiment depicted in the figures.

### BRIEF DESCRIPTION OF THE FIGURES

There are:

FIG. 1 shows an embodiment of an apparatus according to the invention in a perspective and schematic illustration;

FIG. 2 shows another embodiment of an apparatus according to the invention in a vertical and schematic cross-sectional illustration; and

FIG. 3 shows an electromicroscopic image of microorganisms attached to an embodiment of an apparatus according to the invention.

### DETAILED DESCRIPTION OF SEVERAL EXEMPLARY EMBODIMENTS

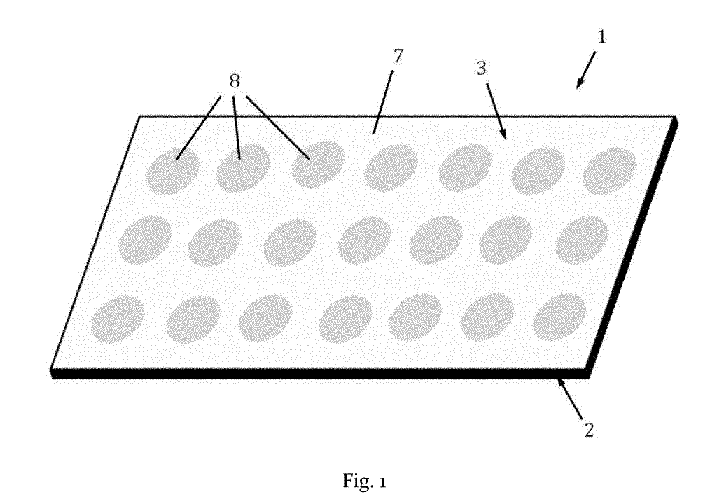

In FIG. 1, an embodiment of an apparatus 1 according to the invention in a perspective and schematic illustration is shown. For example, microorganisms 9 (e.g. spores) can be adhered to the apparatus 1 to examine a sterilisation effect. For example, spores of the family Bacillaceae or Clostridiaceae can be adhered. The apparatus 1 comprises a carrier element 2. The carrier element 2 is, for example, plate-shaped. For example, a glass substrate is used as material for the carrier element 2. The carrier element 2 has 21 microorganism-populations 8, which are attached to activated sub-areas 4 on the surface 3 of the carrier element 2. The individual bondable sub-areas 4 are spaced away from each other at even distances. The individual bondable sub-areas 4 have no mutual contact points through the physical distance present among each other. The microorganism-populations 8 are respectively spaced away from each other.

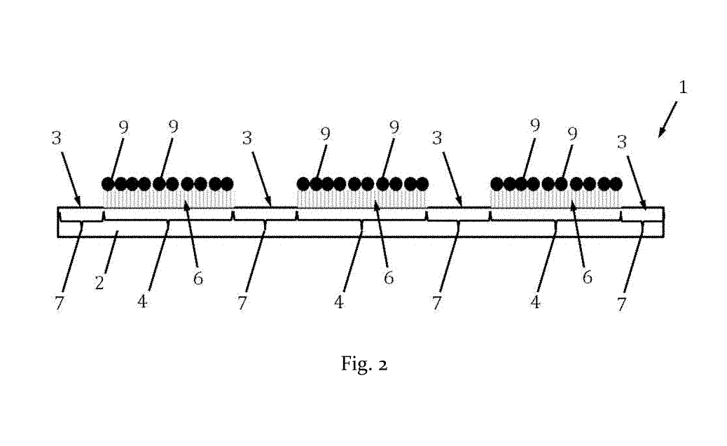

The apparatus 1 comprises a bonding agent (c.f. reference number 6 in FIG. 2), wherein the bonding agent 6 is arranged on a bondable sub-area 4 of the surface 3 respectively and therefore functionalises this bondable sub-area 4. Functionalised sub-areas 5 are set up and/or formed. In particular, the bonding agent 6 fully covers the respective bondable sub-area 4, wherein the bonding agent 6 bonds to the bondable sub-area 4 of the surface 3 of the carrier element 2. In addition, the bonding agent 6 provides free adhesive spots for the adhesion of microorganisms 9 (e.g. spores). In this way, between the respective bondable sub-area 4 of the surface 3 of the carrier element 2 and the microorganisms 9 to be adhered, a stable intermediate bond can be established.

The illustrated embodiment of an apparatus 1 according to the invention comprises at least one population of microorganisms 8, at hand a spore population. On the apparatus 1, for example, in each case a spore population 8 is adhered onto one of the 21 bondable sub-areas 5 of the surface 3 of the carrier element 2, which are functionalised with the bonding agent 6. The microorganisms (e.g. spores 9) of the respective population 8 are chemically purified at present. In the case of chemical purification according to the invention, a purification of the microorganisms (e.g. spores 9) takes place, where bonded excess biological material is removed from the microorganisms (e.g. spores 9). In this way, an individual chemically purified microorganism (e.g. spores 9) has almost a single-cell character. Microorganisms (e.g. spores 9) chemically purified in this way are then adhered to one of the functionalised sub-areas 5. Due to the single-cell character of the respective microorganisms (e.g. spores 9) of a population 8, the microorganisms (e.g. spores 9) are adherable in a high areal density onto the apparatus 1.

All bondable sub-areas 4 of the carrier element 2 primarily have the same size and shape. Due to the identical size and shape, the same individual number of microorganisms are adherable to each of the bondable sub-areas 4, in this case spores 9. At present, the individual microorganisms (e.g. spores 9) of the respective population 9 are electrostatically adhered to the carrier element 2 in a way pertaining to the invention.

FIG. 2 is another embodiment of an apparatus (e.g. apparatus 1 according to FIG. 1) in a vertical and schematic cross-sectional illustration is shown.

Three bondable (e.g. due to a corresponding activation of the area of the surface 3) sub-areas 4 on the surface 3 of the carrier element 2 of the apparatus 1 are shown as a cross-sectional view. The bondable sub-areas 4 are spaced away from each other. The surface 3 of the carrier element 2 between the bondable sub-areas 4 is not respectively activated. It has non-activated sub-areas 7 between the bondable sub-areas 4, wherein this area is characterized with the curly bracket.

A bonding agent 6 is respectively arranged or attached to the bondable sub-areas 4 of the surface 3 of the carrier element 2, wherein the respective bondable sub-areas 4 are completely wetted with the bonding agent 6. The bonding agent 6 attaches to the bondable sub-areas 4 of the surface 3 of the carrier element 2. In addition, the bonding agent 6 provides adhesive spots at which microorganisms (e.g. spores 9) can adhere to. The bonding agent is schematically shown by vertical lines, which should make clear that the bonding agent 6 can establish a stable intermediate bond between microorganisms (e.g. spores 9) and a bondable sub-area 4 of the surface of the carrier element. On the one hand, the bonding agent 6 attaches to the bondable sub-area 4 of the surface 3 of the carrier element 2 and, on the other hand, provides free adhesive spots for microorganisms (e.g. spores 9).

Only a few of the microorganisms that adhere to the bonding agents 6 (e.g. spores 9) are schematically shown. In real, approximately 10⁶microorganisms (e.g. spores 9) are adhered to the surface 3 of the carrier element 2 as a population 8 for each bondable sub-area 5 that is functionalised with the bonding agent 6. The adhesion of microorganisms (e.g. spores 9) of a population 8 primarily takes place without the spores covering each other as is also schematically shown in FIG. 2. The microorganisms (e.g. spores 9) are adhered to the apparatus 1 in a single layer or almost monolayer.

According to the invention, on the one hand, this is made possible by the chemical purification of the microorganisms (e.g. spores 9), wherein the microorganisms (e.g. spores 9) are prepared accordingly before the adhesion. During the chemical purification of the microorganisms (e.g. spores 9), biological material attached to them, in particular, cell debris is removed. This is excess and is not required to examine a sterilisation effect. The intensive chemical purification prepares microorganisms (e.g. spores 9) to be adhered in such a way that these have a single-cell character. The advantage here is that the bonding of the microorganisms (e.g. spores 9) to the functionalised sub-area 5 of the carrier element 2 is higher than a possible bonding of the microorganisms (e.g. spores 9) to a non-bondable sub-area 7 of the carrier element 2 and/or to other microorganisms (e.g. spores 9). In particular, a mutual bonding of microorganisms (e.g. spores 9) among one another can lead to a clumping of the microorganisms (e.g. spores 9), wherein the microorganisms can mutually protect themselves from being exposed to sterilisation conditions.

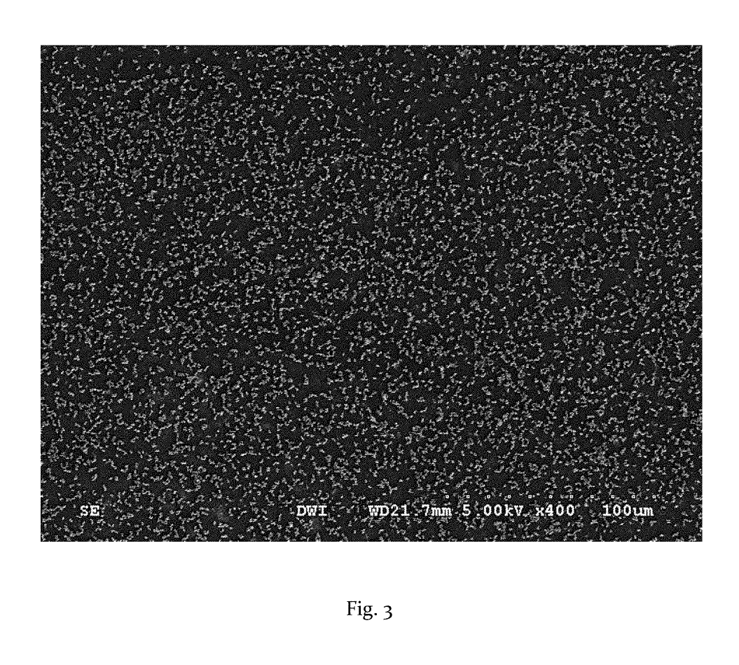

In FIG. 3, an electromicroscopic image of microorganisms attached to an embodiment of an apparatus according to the invention is shown. The illustrated microorganisms are of a strain of Geobacillus stearothermophilus ATCC 7953, and were adhered to an apparatus by means of an ionic bonding agent and attached according to at least one of the aforementioned aspects. An exemplary counting and extrapolation of adhered microorganisms showed 1.9×10⁵ CFU/mm² (CFU: colony-forming unit, corresponds to the number of microorganisms on a surface of 1 mm²). Furthermore, it can be seen that the adhered microorganisms are adhered in high areal density and primarily without covering each other in a single layer.

The microorganisms shown in FIG. 3 were highly purified before the adhesion to an apparatus, wherein the following purification steps have been performed to obtain the high level of purification in the following way:
Manufacturing potassium phosphate buffer (3 M):
16.89 g KH₂PO₄+30.66 g K₂HPO₄ on 100 mL WFI, pH=7.1.
Manufacturing of System Y:
11.18 g PEG 4000+34.1 mL phosphate buffer
Add approximately 10 ml of the spore suspension into system Y
Shake in Falcon tubes for 15 minutes, then centrifuge for 2 min at 1500 rcf
Take the upper ⅔ of the upper phase

Replace PEG at this stage with WFI by means of centrifugation (Wash 3 times; 6000 rcf, 30 min, 10° C.)

The advantage of the solution according to the invention with respect to known apparatuses is that due to the adhesion of at least two populations of microorganisms, meaning multiple populations on only one carrier element, a joint exposure of these multiple populations to sterilisation conditions is possible. Thereby, the predominant sterilisation conditions are not only almost identical for the individual microorganisms of the population, but also for respective populations of microorganisms due to the very near spatial proximity of their arrangement to each other. The bonding of microorganisms according to the invention on the carrier element allows for the microorganism load that should be inactivated according to the requirements at hand, to vary according to the process. Higher resistance or a greater number of used microorganisms allows for a meaningful verification of highly effective processes; a greater number of populations for each carrier element enables a more precise statistical evaluation.

The exemplary embodiments described within this specification should be understood both individually as well as in combination with each other as disclosed. In particular, the description of a feature attributed to an embodiment should also—provided that the contrary has not been explicitly declared—not be understood that the feature is crucial or essential for the function of the embodiment. The sequence of the process steps explained within the specifications in the individual flowcharts is not mandatory, alternative sequences of the process steps are also conceivable. The process steps can be implemented in various ways, in this way, and implementation into software (via programme instructions), hardware or a combination of both to implement the process steps is conceivable. The terms used in the patent claims, such as “comprise”, “have”, “contain”, “include” and the like do not rule out other elements or steps. Both the case of “partly” as well as the case “completely” fall under the formulation “at least partly”. The wording ‘and/or’ is to be understood that both the alternative and the combination should be disclosed, so “A or B” means (A) or (B) or (A and B). A plurality of units, persons or the like means multiple units, persons or the like in the context of these specifications. The use of the indefinite article does not rule out a plural. A single set-up can implement the functions of a plurality of the units or set-ups mentioned in the claims. References specified in the patent claims must be not be viewed as restrictions on the means and steps used.

The exemplary embodiments illustrated and described in the figures are used only by way of explanation of the invention and are not to be taken in a limiting sense.

1. An apparatus for examining a sterilisation effect, comprising:
a carrier element, wherein at least two sub-areas of the surface of the carrier element are bondable and wherein the bondable sub-areas are spaced away from each other;
a bonding agent, wherein the bondable sub-areas of the surface due to the arrangement of the bonding agent to these are functionalisable, wherein the bonding agent, on the one hand, binds to the bondable sub-area of the surface, and on the other hand, due to the respective size of the bondable sub-area, provides free adhesive spots for microorganisms to be adhered so that a stable intermediate bond is creatable between the bondable sub-area of the surface of the carrier element and microorganisms to be adhered, and
a population of microorganisms, wherein the microorganisms of the population are purified, wherein excess biological material attaching to the microorganisms is removed and the respective microorganisms have an almost single-cell character,
wherein a purified population of microorganisms is adherable to one of the functionalised sub-areas of the surface in such a way that the microorganisms are adhered in a high areal density and primarily without mutual covering within a single layer.

2. The apparatus according to claim 1, characterized in that the high areal density of the microorganisms, which are adherable to the functionalised sub-areas of the surface of the carrier element, corresponds to a value, the standard deviation of which is approximately 10% or less with reference to a areal density of approximately 10⁶ microorganisms per 5 mm².

3. The apparatus according to claim 1, characterized in that the adhesion of microorganisms in the single layer occurs in such a way, wherein approximately 10% or less of the microorganisms are covering each other.

4. The apparatus according to claim 1, characterized in that the at least two bondable sub-areas of the surface of the carrier element are activated.

5. The apparatus according to claim 1, characterized in that the respective area of the bondable sub-areas on the surface of the carrier element is primarily identical in size.

6. The apparatus according to claim 1, characterized in that the bonding agent connects to the bondable sub-areas of the surface of the carrier element and microorganisms to be attached bond to the adhesive spots provided by the bonding agent in an electrostatic or covalent manner.

7. The apparatus according to claim 1, characterized in that the bondable sub-areas of the surface of the carrier element primarily free of cavities.

8. The apparatus according to claim 1, characterized in that the carrier element is primarily plate-shaped, spherical, polyhedral or the like.

9. The apparatus according to claim 1, characterized in that the bondable sub-areas of the surface of the carrier element are elevated in relation to the non-bondable sub-area of the surface of the carrier element.

10. The apparatus according to claim 1, characterized in that the microorganisms are spores, in particular, endospores belonging to the family Bacillaceae or Clostridiaceae.

11. A method for manufacturing an apparatus according to claim 1, comprising the following method steps:
Providing a carrier element, wherein at least two sub-areas of the surface of the carrier element are bondable and wherein the bondable sub-areas care spaced away from each other.
Functionalisation of the bondable sub-areas of the surface of the carrier element with a bonding agent, wherein the bonding agent, on the one hand, binds to the bondable sub-area of the surface, and on the other hand, due to the size of the bondable sub-area, provides free adhesive spots for microorganisms to be adhered so that a stable intermediate bond is created between the bondable sub-area of the surface of the carrier element and the microorganisms to be adhered;
Purification of microorganisms, wherein excess biological material attaching to the microorganisms is removed so that the microorganisms have an almost single-cell character; and
Adhesion of the microorganisms, wherein the purified microorganisms are applied to the sub-areas functionalised with the bonding agent and the microorganisms adhere to the adhesive spots provided by the bonding agent so that the microorganisms are adhered in a single layer without primarily covering each other and in a high areal density.

12. The method according to claim 11, characterized by the following process step:
Purification of the carrier element, wherein secondarily attached microorganisms and/or microorganisms attached on a non-bondable sub-area are removed from the carrier element.

13. The method according to claim 11, characterized in that the at least two bondable sub-areas are activated prior to functionalisation, wherein the activation preferably takes place by means of a plasma treatment.

14. The method according to claim 11, characterized in that the adhesion of the microorganisms takes place by applying microorganisms to be adhered in excess onto the at least one functionalised sub-area of the surface of the carrier element.

15. A method, comprising the step of the apparatus according to claim 1 to examine a sterilisation effect and/or as a bioindicator.
